(19) United States
(12) Patent Application Publication
(10) Pub. No.: US 2026/0204450 A1
(43) Pub. Date: Jul. 16, 2026

(54) DOSE DISTRIBUTION CALCULATION SYSTEM, PROGRAM, AND DOSE DISTRIBUTION CALCULATION METHOD

(71) Applicant: HITACHI HIGH-TECH CORPORATION, Tokyo (JP)

(72) Inventors: Shusuke HIRAYAMA, Tokyo (JP); Takahiro YAMADA, Tokyo (JP)

(21) Appl. No.: 19/137,288

(22) PCT Filed: Jul. 5, 2023
(86) PCT No.: PCT/JP2023/025032

§ 371 Date: Jun. 9, 2025

(30) Foreign Application Priority Data
Mar. 29, 2023 (JP) 2023-052546

(51) Int. Cl. G21K 1/04; A61N 5/10

(52) U.S. Cl. CPC G21K 1/046; A61N 5/1031; A61N 5/1045; A61N 2005/1034

(57) ABSTRACT

A dose distribution calculation system 113 for calculating a dose distribution of radiation irradiated through a multi-leaf collimator includes: an MLC transport calculation unit 120 for calculating transmission paths through the multi-leaf collimator on two projection planes for each of particles of the radiation to calculate transmittance for each of the particles; and a dose distribution calculation unit 121 for calculating the dose distribution using the transmittance. This configuration provides a dose distribution calculation system, a program, and a dose distribution calculation method that are capable of efficiently obtaining a dose distribution calculation result with high accuracy.

### TECHNICAL FIELD

The present invention relates to a dose distribution calculation system, a program, and a dose distribution calculation method that are suitably used in a radiotherapy system for irradiating and treating an affected area such as a tumor with radiation.

### BACKGROUND ART

PTL 1 describes steps of: defining a three-dimensional geometry of a collimator device defining an aperture configured to allow a radiation beam to pass through the aperture; projecting the collimator along the radiation beam into a two-dimensional geometry on a plane; calculating a dose opacity of the collimator device at a position adjacent to an opening based on a three-dimensional shape of the collimator device; and calculating a transport of the radiation beam through the collimator device based on a two-dimensional shape projected on the plane and using the dose opacity of the collimator device at the position adjacent to the opening.

NPL 1 describes dose distribution calculation of intensity modulated radiotherapy in which not only influence of a shape of a multi-leaf collimator, such as leaf transmission, inter-leaf leakage, a rounded leaf tip, and an effect of a leaf sequence, and beam divergence, but also energy fluctuation of the entire field is taken into a model.

NPL 2 describes a runsport-based multi-leaf collimator particle transport model that is used in Monte Carlo dose distribution calculations and that is accurate, fast and efficient, and thus the model is applicable to iterative intensity modulated radiotherapy dose distribution calculations.

### CITATION LIST

### Patent Literature

PTL 1: US 2020/0298018 A

### Non-Patent Literature

NPL 1: R. F. Aaronson et al., “A Monte Carlo based phase space model for quality assurance of intensity modulated radiotherapy incorporating leaf specific characteristics”, Med. Phys. 29 (12), 2952-2958 (2002).
NPL 2: J. V. Siebers et al., “A method for photon beam Monte Carlo multileaf collimator particle transport”, Phys. Med. Biol. 47, 3225-3249 (2002).

### SUMMARY OF INVENTION

### Technical Problem

Radiation therapy is a local therapy in which cancer cells are killed by irradiating a cancer lesion with radiation to damage DNA of the cancer cells. Examples of the radiation species to be irradiated include X-rays and particles such as protons and carbon ions, and X-rays are most used among them.

The radiotherapy using X-rays is performed by a high-precision irradiation method in which dose concentration is improved by irradiating a target with X-rays from multiple irradiation directions, such as intensity modulated radiation therapy (IMRT), volumetric modulated arc therapy (VMAT), and dynamic wave arc (DWA) in which continuous non-coplanar irradiation is performed in a wavy trajectory.

The VMAT enables a high dose to be applied to a tumor while reducing a dose to normal tissues by performing continuous rotational irradiation while continuously modulating three parameters of a gantry angle, an opening shape of a multi-leaf collimator (MLC), and a dose rate.

The radiotherapy uses instrument parameters (such as an opening shape of a collimator) of a treatment apparatus, and irradiation parameters such as an irradiation angle, the amount of irradiation for each irradiation angle, and energy, the parameters being determined by simulating dose distribution in a patient body using a treatment planning software based on a patient CT image and a prescription dose set by a doctor.

Here, the determined irradiation parameters and the dose distribution serving as a calculation basis of the irradiation parameters are referred to as treatment planning. When an irradiation device irradiation with a beam based on the treatment planning, a desired dose distribution can be formed in an affected area.

The treatment planning used for treatment is checked to ensure health of the treatment plan by checking whether the device can perform irradiation to form the dose distribution as in the treatment planning before irradiating the patient. This is called patient QA. The most common method performed in the patient QA is to perform radiation according to the treatment planning by actually irradiating a homogeneous phantom such as an individual phantom to actually measure a dose distribution.

Through the patient QA, clinical staff performs the following: (1) verification of calculation accuracy of a dose distribution calculation algorithm of the treatment planning software; (2) verification of influence on the dose distribution due to an irradiation error of the irradiation device in the homogeneous phantom; and (3) verification of validity of device operation for the treatment planning.

The items (1) and (2) are verified by comparing actual measurement results with calculation results of the treatment planning software. Treatment facilities set evaluation items and criteria for the patient QA for each facility, and after each evaluation item is checked that corresponding one of the criteria is satisfied, the facility can be used for the treatment plan. When deviation from the criteria is checked, the patient QA is performed again or treatment planning is created again.

The radiotherapy is performed by repeating irradiation of an affected area once a day for several days to several weeks. Thus, the tumor may be enlarged or reduced, or internal structure around the tumor may be changed during a treatment period.

For such a change having occurred, need for adaptive treatment has increased, the adaptive treatment being performed by creating treatment planning again and performing treatment using the treatment planning created again.

Examples of the adaptive treatment include treatment called online adaptive treatment in which treatment planning is created again immediately before treatment while a patient lies on a treatment bed.

The online adaptive treatment optimizes a dose in accordance with a patient body shape different for each treatment day such as a state of the nasal cavity and the intestine. A dose distribution is optimized for each treatment day, so that a region as a margin outside a target to be irradiated can be reduced, and thus damage to normal tissues can be reduced.

Unfortunately, the online adaptive treatment has various development problems such as a great increase in treatment time due to increase in the number of processes to be performed during treatment such as re-creation of treatment planning. One of the problems is the patient QA for the online adaptive treatment.

The online adaptive treatment requires the patient QA of replanned treatment planning to be performed in a state where the patient lies on a couch, and thus the patient QA based on actual measurement by actual irradiation, which has been previously performed, is less likely to be performed.

Thus, a method has been proposed in recent years, the method being for checking whether a planned dose distribution can be formed in a CT image or a homogeneous phantom using an independent dose distribution calculation engine using a high-precision dose distribution calculation method such as a Monte Carlo method in a patient QA.

The Monte Carlo method is for calculating a dose distribution by reproducing individual radiation behavior stochastically using random numbers, and repeating this reproduction many times (several million times or more) to estimate an average behavior of the radiation. Calculation of a dose distribution of X-ray therapy requires enormous calculation time for the Monte Carlo simulation that is performed all around a process from a stage in which electrons accelerated by an accelerator are irradiated to a tungsten target to generate X-rays, the X-rays passing through an MLC to form a radiation field, and the X-rays with the radiation field formed are irradiated to a patient to give a dose of the X-rays to the patient.

Thus, the calculation is typically performed by dividing the process into two processes including: (A) a process until the radiation field is formed by the MLC; and (B) a process in which the X-rays with the radiation field formed are transported inside the patient.

The present invention relates to the calculation of the radiation field formation using the MLC in the former process (A).

For the VMAT irradiation or the like, the process of the radiation field formation is calculated using an analytical method or a quasi-analytical method to efficiently perform transport calculation in the process of the radiation field formation using the MLC because a leaf shape is complicatedly changed in accordance with rotation of a gantry.

PTL 1 and NPL 1 described above each describe a calculation method of a process of irradiation field formation using an analytical method. These documents each disclose a method for calculating a dose distribution by calculating a transmittance map of beams obtained by projecting a three-dimensional MLC shape from an irradiation direction of the beams in consideration of influence of an MLC shape using the transmittance map. When a treatment plan of the VMAT is created, the MLC shape for each irradiation angle is determined by optimization calculation by discretizing continuous irradiation angles into a finite number of irradiation angles (e.g., a gantry angle separated by two degrees) to reduce calculation cost. Thus, analytical calculation methods are widely used in a process of creating treatment planning.

NPT 2 discloses a calculation method for a process of radiation field calculation using a quasi-analytical method.

The quasi-analytical method considers influence of an MLC shape by steps of: generating particle species information (information on a type, a position, energy, and a traveling direction of particles) upstream of the MLC, the information being calculated or modeled in advance using the Monte Carlo simulation; calculating a passage length in the MLC for each of particles using the particle species information as an input; and setting transmittance calculated from the calculated passage length as a weight of the particle.

Here, the number of particles used in the Monte Carlo simulation is several millions or more, and the quasi-analytical method needs to repeat the calculation of the passage length in the MLC several times to calculate the transmittance for each of particles, so that calculation speed and calculation accuracy of the calculation method of the passage length in the MLC are extremely important.

NPL 2 uses a method for calculating a path length of a particle in an MLC based on not only a drive direction of a leaf of the MLC in consideration of a shape of the leaf, but also a placement direction of the leaf that uses a thickness represented by injection positions of particles on a leaf upper surface and a leaf middle surface. This method may deteriorate in calculation accuracy of particles passing through a region having different leaf positions between adjacent leaves when the particles go out of the leaf between the leaf upper surface and the leaf middle surface or between the leaf middle surface and a leaf lower surface.

Although examples of a method for calculating the passage length with high accuracy include a method using three-dimensional ray trace calculation, this method causes increase in calculation cost and is less likely to calculate the MLC passage length with high accuracy in a short time.

The present invention provides a dose distribution calculation system, a program, and a dose distribution calculation method that are capable of efficiently obtaining a dose distribution calculation result with high accuracy.

### Solution to Problem

The present invention includes a plurality of means for solving the above problems, and an example thereof is a dose distribution calculation system for calculating a dose distribution of radiation irradiated through a multi-leaf collimator, the system including: a transport calculation unit for calculating transmission paths through the multi-leaf collimator on two projection planes for each of particles of the radiation to calculate transmittance for each of the particles; and a dose distribution calculation unit for calculating the dose distribution using the transmittance.

### Advantageous Effects of Invention

According to the present invention, a dose distribution calculation result with high accuracy can be efficiently obtained. Problems, configurations, and effects other than those described above will be clarified by the following description of embodiments.

### BRIEF DESCRIPTION OF DRAWINGS

FIG. 1 is a general configuration diagram of a radiotherapy system of the present embodiment.

FIG. 2 is a flowchart showing steps of calculating a dose distribution by a dose distribution calculation system of the present embodiment.

FIG. 3 is a flowchart showing an MLC transport calculation steps by the dose distribution calculation system of the present embodiment.

FIG. 4 is a flowchart showing steps of calculating transmittance of an MLC for each of particles by the dose distribution calculation system of the present embodiment.

FIG. 5 is a shape example on each projection plane of the MLC in the dose distribution calculation system of the present embodiment.

FIG. 6 is a conceptual diagram for illustrating a calculation process of an MLC passage length in the dose distribution calculation system of the present embodiment.

FIG. 7 is a flowchart showing another example of the MLC transport calculation steps by the dose distribution calculation system of the present embodiment.

### DESCRIPTION OF EMBODIMENTS

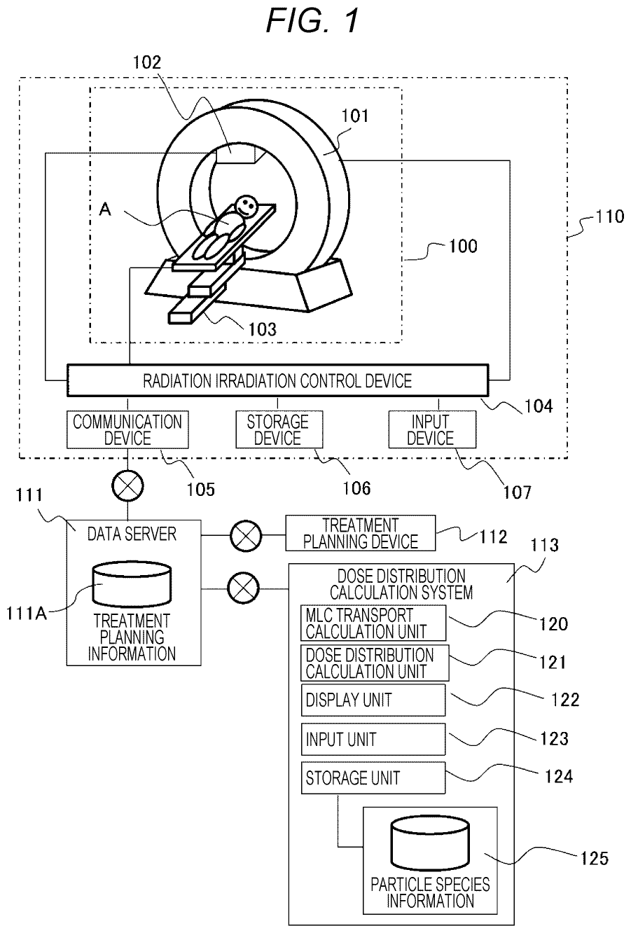

An embodiment of a dose distribution calculation system, a program, and a dose distribution calculation method of the present invention will be described with reference to FIGS. 1 to 7. The drawings used herein each show the same or corresponding components that are denoted by the same or similar reference numerals, and duplicated description of these components may not be described.

Although the present invention can be suitably applied to an X-ray irradiation system using X-rays as radiation to be used, the present invention can also be suitably applied to a particle beam irradiation system using particles such as protons and carbon ions besides the X-rays. In the following embodiment, an X-ray irradiation system will be described as an example.

First, a general configuration of an X-ray therapy system of the present invention will be described with reference to FIG. 1. FIG. 1 is a diagram schematically illustrating the general configuration of the X-ray therapy system.

The X-ray therapy system illustrated in FIG. 1 includes an X-ray irradiation system 110, a data server 111, a treatment planning device 112, and a dose distribution calculation system 113.

The X-ray irradiation system 110 includes an X-ray irradiation device 100, a radiation irradiation control device 104, a communication device 105, a storage device 106, and an input device 107. The X-ray irradiation device 100 includes a ring type gantry 101, an irradiation nozzle 102, and a couch 103.

The X-ray irradiation system 110 is configured such that when an extraction start signal is output from the radiation irradiation control device 104, a linear accelerator in the irradiation nozzle 102 accelerates an electron beam, and X-rays are generated by irradiating tungsten with the accelerated electron beam.

The irradiation nozzle 102 is internally provided with a collimator (referred to below as a multi-leaf collimator (MLC)) including a plurality of plate-shaped shields (referred to below as leaves) disposed left and right, and a position of each leaf is appropriately changed based on an instruction value from the radiation irradiation control device 104 to shape the generated X-rays into a desired distribution.

The irradiation nozzle 102 is also internally provided with a dose monitor that measures an irradiation amount of an X-ray, and the detected measurement value is output to the radiation irradiation control device 104 to be used for control during radiation irradiation.

A bed on which an irradiation target A is placed is referred to as the couch 103. The couch 103 can be moved in directions of three orthogonal axes based on an instruction from the radiation irradiation control device 104, and can be further rotated about each axis. The movement and rotation enable the irradiation target A to be moved to a desired position.

The irradiation nozzle 102 is mounted on the ring type gantry 101, and a gantry rotation angle and a ring rotation angle are set in response to an instruction value from the radiation irradiation control device 104, and the irradiation target A on the couch 103 can be irradiated with the X-ray from an appropriate angle. The rotation center of each of the gantry rotation and the ring rotation is referred to as an isocenter.

The radiation irradiation control device 104 is connected to the ring type gantry 101, the irradiation nozzle 102, the couch 103, the communication device 105, the storage device 106, the input device 107, and the like to control devices in the irradiation nozzle 102, the ring type gantry 101, the couch 103, and the like.

The communication device 105 is connected to the data server 111 through a network to acquire treatment planning data in which irradiation parameters (information on a gantry angle, an irradiation amount, a leaf position, etc.) created by the treatment planning device 112 through the network before irradiation are stored from the data server 111, and stores the irradiation parameters in the storage device 106.

The input device 107 is connected to the radiation irradiation control device 104 to display information on a monitor based on a signal acquired from the radiation irradiation control device 104. The input device 107 also receives an input signal from a medical worker who operates the X-ray irradiation system 110 to transmit various control signals to the radiation irradiation control device 104. Upon receiving an instruction to start irradiation with radiation through the input device 107, the radiation irradiation control device 104 starts the irradiation based on the irradiation parameters stored in the storage device 106.

The dose distribution calculation system 113 calculates a dose distribution of radiation irradiated through the multi-leaf collimator, and includes an MLC transport calculation unit 120, a dose distribution calculation unit 121, a display unit 122, an input unit 123, and a storage unit 124. The dose distribution calculation system 113 is connected to the data server 111 through a network to acquire treatment planning information for calculating the dose distribution.

The MLC transport calculation unit 120 calculates a passage length in the MLC for each of particles using the particle species information on an upper surface of the MLC stored in the storage unit 124 as an input, and sets transmittance calculated from the passage length as a weight of the particle to generate particle species data downstream of the MLC. The MLC transport calculation unit 120 is preferably responsible for performing a transport calculation procedure and transport calculation steps.

In the present embodiment, the MLC transport calculation unit 120 calculates transmission paths through the multi-leaf collimator on two projection planes for each of particles of the radiation to calculate transmittance for each of the particles. Here, the two projection planes can serve as a projection plane in a leaf placement direction of the multi-leaf collimator and a projection plane in a leaf moving direction. When the transmittance is calculated, the transmittance can be acquired by acquiring a path length from a common range of transmission paths calculated on the two projection planes for each of particles of the radiation.

The MLC transport calculation unit 120 also can perform steps of: interpolating device information at each control point using a common interpolation parameter set for each of particles of the radiation; setting a different device parameter for each of the particles of the radiation; generating particle species information in consideration of the device information that continuously changes; and acquiring a path length using the generated particle species information.

The MLC transport calculation unit 120 can further generate Compton photons as particles and calculate transmittance of the Compton photons.

The dose distribution calculation unit 121 calculates a dose distribution in a patient body or in a numerical phantom using the particle species data and the transmittance downstream of the MLC as inputs, which are generated by the MLC transport calculation unit 120. Here, the particle species data includes position information, a travel vector, energy, a particle type (X-ray, electron, positron), and a statistical weight for each of particles and photons. The dose distribution calculation unit 121 is preferably responsible for performing the dose distribution calculation procedure and the dose distribution calculation steps.

The display unit 122 is a display that displays the dose distribution calculated by the dose distribution calculation unit 121.

The radiation irradiation control device 104 and the dose distribution calculation system 113 each include a central processing unit (CPU) and a memory connected to the CPU.

Control processing of operation to be performed may be integrated into one program, may be divided into a plurality of programs, or may be a combination of the programs.

A part or all of a program held by each device may be implemented by dedicated hardware or may be modularized.

Various programs may be installed in each device using a program distribution server or an external storage medium, or an existing device may be updated for the various programs.

Each device may be an independent device and connected through a wired or wireless network, or two or more devices may be integrated.

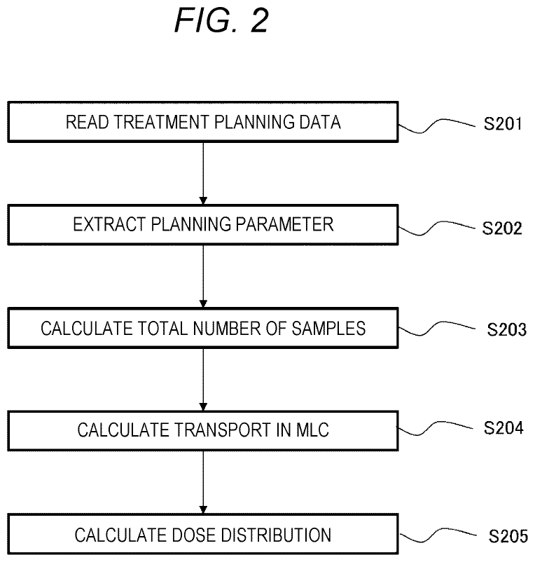

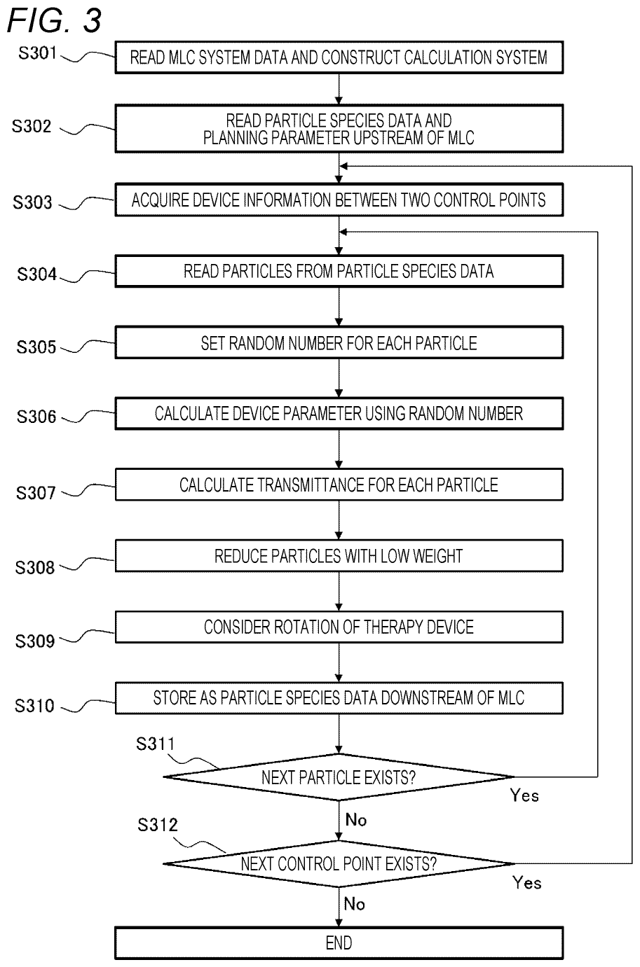

Subsequently, an embodiment of calculating a dose distribution in a patient body using the dose distribution calculation system 113 of the present embodiment will be described. FIG. 2 shows an entire flow of dose distribution calculation, and FIG. 3 shows a flow of a section of transport calculation in the MLC, which is characteristic processing of the present invention.

As shown in FIG. 2, first, the dose distribution calculation system 113 acquires treatment planning data for verifying the dose distribution from the data server 111 (S201). At this time, an operator can select the treatment planning data to be read by the dose distribution calculation system 113 from treatment planning information 111A in the data server 111 using the input unit 123, or the dose distribution calculation system 113 can automatically read the treatment planning data by searching the treatment planning information 111A in the data server 111 for the treatment planning data read by the radiation irradiation control device 104.

Subsequently, the MLC transport calculation unit 120 of the dose distribution calculation system 113 extracts planning parameters to be used for dose distribution calculation and MLC transport calculation from the treatment planning data read in step S201, and stores the extracted planning parameters in the storage unit 124 (S202).

Subsequently, the MLC transport calculation unit 120 calculates a total number of samples at a position upstream of the MLC using the planning parameters of the treatment planning data read in step S202 (S203). Here, the number of samples is the number of particles used for dose distribution calculation, and an irradiation amount of an X-ray is determined by the MU value of the dose monitor installed in the irradiation nozzle in the X-ray irradiation device 100. Thus, the number of particles per MU value is often used. An example is here described in which the number of samples [particles/(MU/Gy)] calculated as per unit dose at a normalization point is used.

The MLC transport calculation unit 120 acquires a dose value and MU value information at the normalization point from the treatment planning information to calculate the MU value per unit dose [MU/Gy] at the normalization point. The number of particles per MU value per unit dose [particles/(MU/Gy)] is stored in advance in the storage unit 124, and the total number of samples is calculated by multiplying the MU value per unit dose at the normalization point by the number of particles per MU value per unit dose. Here, a value designated by the operator using the input unit 123 may be used as the number of particles [particles/(MU/Gy)] per MU value per unit dose.

Subsequently, the MLC transport calculation unit 120 reads particle species data upstream of the MLC, and performs transport calculation in the MLC to generate the particle species data downstream of the MLC (S204).

After the transport calculation is completed, the dose distribution calculation unit 121 of the dose distribution calculation system 113 reads the particle species data downstream of the MLC generated by the MLC transport calculation unit 120 to perform dose distribution calculation (S205). The dose distribution calculation is preformed to calculate a dose Dᵢ of a voxel i according to Expression (1) below where a statistical weight of a particle k as an input is sₖ and a dose given to the voxel i by the particle k is d_{i,k}.

$${D}_{i} = C \times \sum {w}_{k} \times {d}_{i , k} \qquad ( 1 )$$

Here, C is a correction coefficient for converting a dose calculated by the number of calculation samples into a dose given by an actual number of particles.

The present invention is characterized in the method for calculation in the section of the transport calculation in the MLC of step S204. FIG. 3 shows a flow diagram of the transport calculation in the MLC.

Here, an example is described in which the transportation calculation in the MLC is performed for treatment planning of continuous rotation irradiation represented by the VMAT or the DWA.

The continuous rotation irradiation is an irradiation method in which irradiation is performed while continuously modulating three parameters of an irradiation angle (gantry angle, irradiation ring angle), an MLC shape, and a dose rate. When treatment planning using the continuous rotation irradiation is created, the treatment planning is created under conditions where control points of the irradiation device are generated by discretizing irradiation angles at equal intervals to reduce a calculation load at the time of creating the treatment planning. Thus, device information, such as a position of leaf that continuously changes, is less likely to be considered in calculation.

In contrast, the present invention enables particle data to be generated in consideration of device information that continuously changes by setting a different device parameter for each of particles by interpolating the device information at each control point using an identical quasi-random number that is a common quasi-random number set for each of the particles. Consequently, a dose distribution can be calculated more accurately than before. This is one of the features of the present invention.

Although a quasi-random number used as an interpolation parameter will be described, a random number or a uniform random number can be used besides the quasi-random number.

As shown in FIG. 3, the MLC transport calculation unit 120 first reads systematic data on the multi-leaf collimator used in the X-ray irradiation device 100 to construct a calculation system (S301).

The multi-leaf collimator includes a plurality of plate-shaped shielding objects (referred to below as leaves) that can be driven in one axial direction and that are disposed in a line, in which a direction in which the leaves are driven is referred to as a leaf moving direction, and a placement direction of the leaves in which the leaves are disposed is referred to as a leaf placement direction.

In the leaf placement direction, a frame is placed outside the leaves, and has a role of shielding a dose outside a radiation field. As described later, the present invention allows transport of each of particles in the multi-leaf collimator to be calculated using projection plane information on each leaf and the frame in the leaf placement direction of, and projection plane information thereon in the leaf moving direction. Thus, the system information read here is the projection plane information on each leaf and the frame not only in the placement direction but also in the driving direction.

Subsequently, the MLC transport calculation unit 120 reads a particle species information database 125 upstream of the MLC stored in the storage unit 124, and acquires particle data upstream of the MLC, the particle data being an input of the MLC transport calculation (S302).

Subsequently, the MLC transport calculation unit 120 acquires the planning parameters acquired in step S202 from the storage unit 124 (S302). The planning parameters of the continuous rotation irradiation includes a leaf position, a gantry angle, an irradiation ring angle, and an irradiation amount between control points, which are described for each control point.

After that, the MLC transport calculation unit 120 acquires device information at two control points and device information between the two control points from the planning parameters to perform the MLC transport calculation in units of inter-control point (S303). The number of samples used in the MLC transport calculation between the control points is allocated in accordance with a dose for each inter-control points.

Subsequently, the MLC transport calculation unit 120 reads particles from the particle species data upstream of the MLC read in step S302 (S304), and sets the quasi-random number t for each of the particles (S305). A value of “t” set in step S305 means a time parameter related to irradiation timing between two control points, and is given by a uniform random number satisfying a relationship of 0<t<1.

Subsequently, the MLC transport calculation unit 120 sets a different device parameter for each of the particles by interpolating device parameters between two control points using the time parameter above to consider the device information that continuously changes (S306). When tⱼ is given to a particle j as a time parameter, a device parameter εⱼ of the particle j is given by Expression (2) below where device parameters at a control point A and a control point B are ε_{A} and ε_{B}, respectively.

$${ε}_{j} = {ε}_{A} + {t}_{j} \times ( {ε}_{B} - {ε}_{A} ) \qquad ( 2 )$$

The device parameters include a gantry angle, an irradiation ring angle, a leaf position of the MLC, and the like, and a different device parameter is set for each of particles using the same parameter tⱼ for each device.

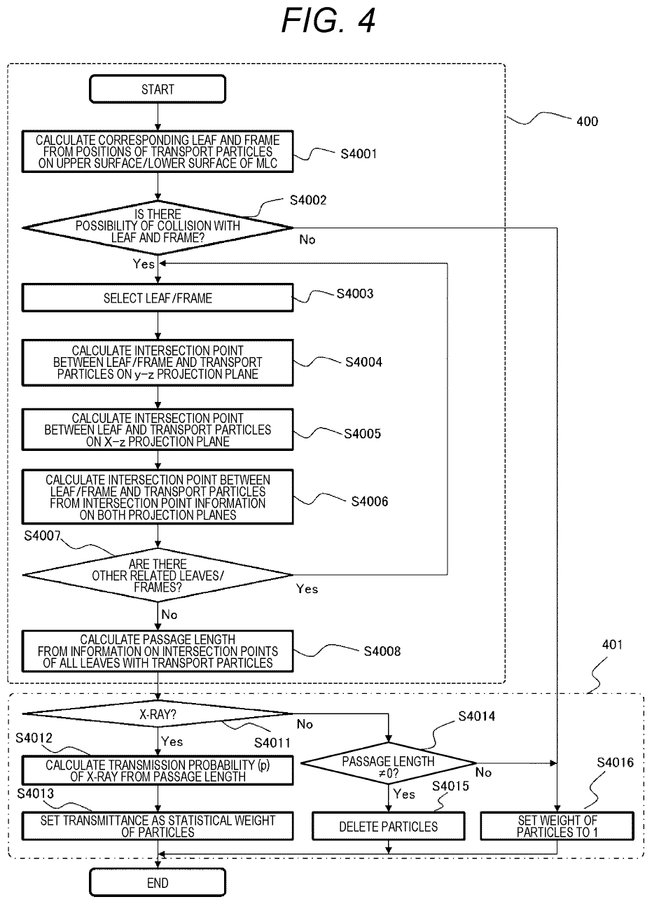

Subsequently, the MLC transport calculation unit 120 calculates a passage length in the MLC for each of particles to calculate transmittance (S307). FIG. 4 shows a calculation flow of the transmittance. With reference to FIG. 4, a method for calculating the transmittance is described.

As shown in FIG. 4, the calculation of the transmittance is divided into two processes of a calculation process 400 of a passage length in the MLC and a transmittance calculation process 401 of calculating the transmittance from the passage length. The MLC transport calculation unit 120 is responsible for performing the two processes.

The present invention has the second feature in the calculation process 400 of the passage length in the MLC in the calculation flow of the transmittance. The present invention allows the passage length in the MLC to be calculated by calculating transmission path information on particles in the MLC on the projection plane in the leaf placement direction and the projection plane in the leaf moving direction of the MLC to acquire a common range of transmission paths on each projection plane. Consequently, the passage length can be efficiently calculated while deterioration in degree of calculation of particles is prevented, the particles passing through a region where leaf positions are different between adjacent leaves.

The calculation process 400 of a passage length in the MLC includes first a step (S4001) in which based on positions of the particles read in step S304 (referred to below as transport particles) on the upper surface and the lower surface of the MLC, leaves and frames, which may intersect with the transport particles, are extracted.

More specifically, a path expression of the transport particles on the projection plane in the drive direction (x direction) of the leaves and the projection plane in the placement direction (y direction) of the leaves is calculated based on travel vectors (vₓ, v_{y}, v_{z}) of the transport particles and positional information (x₀, y₀, z₀). The particles are considered to travel straight in the traveling direction, so that the path expressions of the transported particles in the x direction and the y direction are expressed by linear functions and are expressed by Expressions (3) and (4) below.

$$z = {v}_{z} / {v}_{x} \times x + ( {z}_{0} - {v}_{z} / {v}_{x} \times {x}_{0} ) , \qquad ( 3 )$$

$$z = {v}_{z} / {v}_{y} \times y + ( {z}_{0} - {v}_{z} / {v}_{y} \times {y}_{0} ) , \qquad ( 4 )$$

Based on y coordinates in depths from the upper surface and the lower surface of the MLC calculated using the path expression of the particles in the y direction, a leaf (referred to below as a corresponding leaf) and a frame, which need to be subjected to determination of collision with the particles, are extracted.

Subsequently, when the MLC has a large opening area (a large irradiation field size), some particles collide with the corresponding leaf selected in step S4001, and many particles reach downstream of the MLC without colliding with the leaf or the frame. Thus, the MLC transport calculation unit 120 determines whether a particle collides with the leaf using a leaf position of the corresponding leaf by performing ray tracing calculation only on the particle having a possibility of collision to improve calculation efficiency (S4002).

The particle is transported without colliding with the leaf when an x coordinate xᵤₚ of the particle on the upper surface of the MLC and an x coordinate x_{down} of the particle on the lower surface of the MLC satisfy relationships below, where a maximum value of a left leaf position of the corresponding leaf is xLₘₐₓ, and a minimum value of a right leaf position thereof is XRₘᵢₙ.

$$x {L}_{max} < {x}_{up} < {x}_{Rmin} , and {xL}_{max} < {x}_{down} < {x}_{Rmin} , \qquad ( 5 )$$

Thus, when the relationships described above are satisfied (No in step S402, no possibility of collision), the path length in the MLC is not calculated, and the particle is set to have a statistical weight of 1. In contrast, when Expression (4) is not satisfied (Yes in step S402, with a possibility of collision), processing proceeds to step S403 to start path length calculation.

Subsequently, a leaf or a frame to be calculated is selected from corresponding leaves and frames calculated in step S4001 (S4003), and then an intersection point (excluding a contact point) between a particle and the leaf or the frame on the projection plane in the leaf placement direction is calculated (S4004).

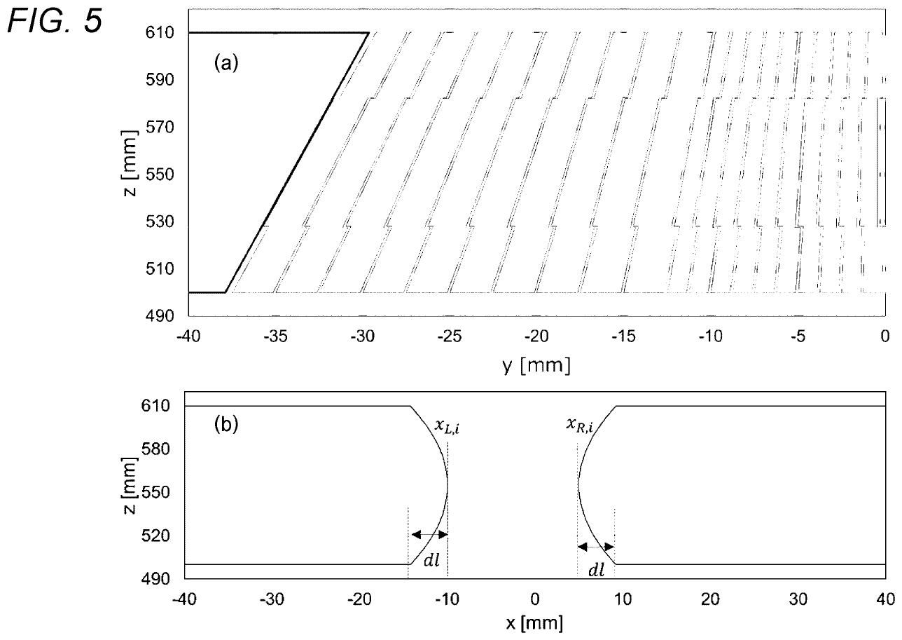

FIG. 5 illustrates an example of a leaf shape and a frame shape of the MLC on each projection plane. FIG. 5 includes a part (a) that illustrates an example of the projection plane in the leaf placement direction, and a part (b) that illustrates an example of the projection plane in the leaf moving direction. As can be found from the part (a) in FIG. 5, the shapes of the leaf and the frame can be expressed by a linear function for an oblique side as with the path expression of particles, and can be expressed by a function form of z=k for other sides. Thus, an intersection point of the path expression of particles with the frame and the leaf can be easily calculated.

Subsequently, an intersection point between a particle and the leaf on the projection plane in the leaf moving direction is calculated (S4005).

The leaf has a shape on the projection plane in the driving direction (x direction), the shape being expressed by an arc shape of a tip of the leaf and the function form of z=k. Equations of the arc shape of the tip of the leaf include an equation of the arc of the tip of the i-th left leaf in the j-th particle, the equation being expressed by Expression (6), and an equation of the arc of the tip of the i-th right leaf in the j-th particle, the equation being expressed by Expression (7), where a leaf intermediate coordinate is Z_{mid}, a radius of an arc part is R₀, a length of the arc part in the x-direction is dl, and leaf positions of the i-th left and right leaves in the j-th particle are x_{L}, i, j and x_{R}, i, j, respectively.

$${( x - ( {x}_{L , i , j} - {R}_{0} ) )}^{2} + {( z - {z}_{mid} )}^{2} = {R}_{0}^{2} , {x}_{L , i , j} - dl < x \leq {x}_{L , i , j} \qquad ( 6 )$$

$${( x - ( {x}_{R , i , j} + {R}_{0} ) )}^{2} + {( z - {z}_{mid} )}^{2} = {R}_{0}^{2} , {x}_{R , i , j} \leq x < {x}_{R , i , j} + dl \qquad ( 7 )$$

Thus, an intersection point of the path expression of particles with the frame can be easily calculated also for an x-z plane.

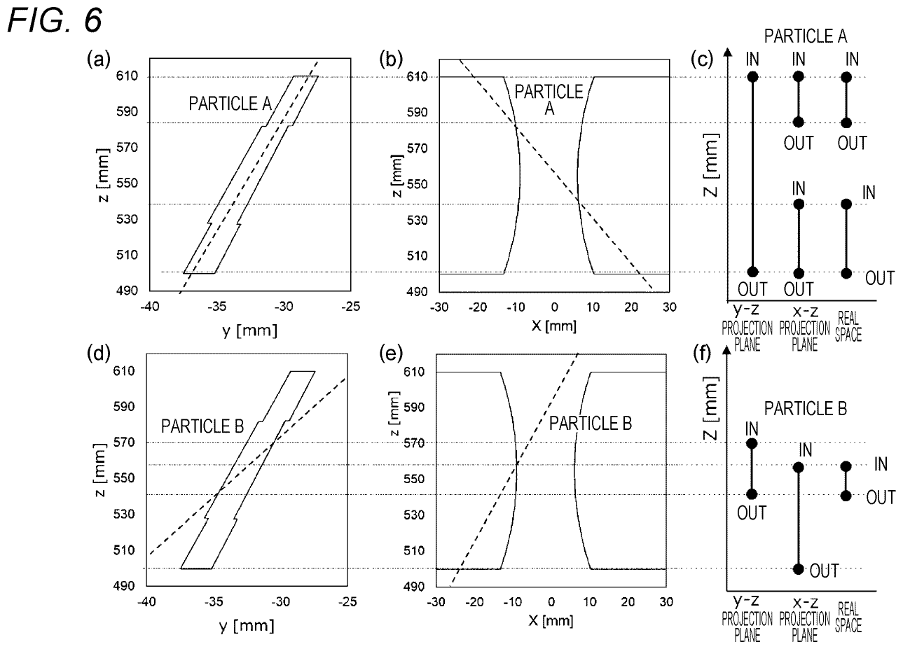

Subsequently, an intersection point with an actual leaf and a transmission path are calculated based on intersection point information calculated on both the projection planes (S4006). This procedure will be described with reference to FIG. 6.

FIG. 6 illustrates a state on each projection plane when a particle (particle A or particle B) is injected on a leaf, intersection point information, and a passage range in the leaf in a z-axis direction.

When intersection points are sorted in descending order by a value of the z axis in the intersection point information on each projection plane created in S4004 and S4005 described above, an odd-numbered intersection point corresponds to a point where the particle enters the leaf, and an even-numbered intersection point corresponds to a point where the particle goes out of the leaf, because an air layer exists between each leaf and frame. Consequently, calculation of the passage range in the leaf in the z-axis direction from the intersection point information can be found.

FIG. 6 includes parts (a) to (c) that illustrate calculation examples of the particle A, in which a transmission path through a leaf is mainly determined by in-out on the projection plane in the leaf moving direction, and parts (d) to (f) that illustrate calculation examples of the particle B, in which “in” is determined on the projection plane in the leaf moving direction and “out” is determined on the projection plane in the leaf placement direction. When the leaf has a shape on the projection plane, the shape being identical to its sectional shape, an intersection point at which the leaf and a particle actually collide, and a transmission path are calculated from a common range of z coordinates of leaf passage ranges on both the projection planes.

The steps from step S4003 to step S4006 are performed until no more related leaf and frame exist (S4007), and the passage length is calculated from information on intersection points of all the leaves with the transport particles (S4008)

The transmittance calculation process 401 is performed to calculate transmittance for each of particles using the passage length in the MLC calculated in the MLC passage length calculation process 400.

First, species of a particle is checked to determine whether the particle is a photon, an electron, or a positron (S4011). When the particle is a photon, transmittance of the X-ray is calculated based on the path length in the MLC (S4012), and the transmittance is set as a statistical weight of the particle (S4013). Transmittance of a photon is generally expressed using an exponential function. Thus, the statistical weight of the X-ray photons set in step S4013 is expressed by Expression (8) below.

$$w = {w}_{0 , j} \times exp ( - μ / ρ ( E ) \times ρ \times t ) , \qquad ( 8 )$$

Here, w_{0, j} means an initial weight of an X-ray photon to be transported, and w_{0, j}=1 for a primary X-ray acquired from a PSD. Additionally, t represents a path length in the MLC calculated in the MLC passage length calculation process 400, ρ represents density of the MLC, and μ/ρ represents a mass attenuation coefficient of a material of the MLC, such as tungsten.

When the particle is an electron or a positron, a path length in the MLC is checked whether to have a value (S4014). When the path length in the MLC has a value (t≠0), the transport particle is deleted because the electron or the positron is shielded by a leaf, (S4015). When the path length has no value (t=0), a statistical weight of the particle is set to 1 (S4016).

After completion of steps S4013, S4015, and S4016, the MLC transport calculation unit 120 shifts the processing to step S308.

Returning to FIG. 2, the MLC transport calculation unit 120 reads data on particle species with statistical weights created in step S204 (steps S301 to S312 in FIG. 3), and calculates a dose distribution using the Monte Carlo method (S205). When there are many particles with small statistical weights in the data on particle species read in step S205, calculation of the dose distribution is greatly reduced in efficiency.

To improve calculation efficiency in step S205, the MLC transport calculation unit 120 thus provides a step (S308) of reducing the number of particles with low weights using the Russian roulette method that is one of examples of a dispersion reduction method illustrated in FIG. 3.

The Russian roulette method is performed such that a cut-off value w_{cut} is set, and particles having a cut-off value w_{cut} or less are reduced with a probability of w/w_{cut} using a uniform random number. In contrast, for particles having survived with a statistical weight equal to or less than w_{cut}, the cut-off value w_{cut} is set as a statistical weight.

Subsequently, after coordinate values of particles at positions downstream of the MLC are calculated, a rotation matrix is created using irradiation angle information (gantry angle information, irradiation ring angle information, and couch angle information) among the device parameters acquired in step S306 (S309).

Subsequently, influence of rotation of a therapeutic device is considered by coordinate transformation applied to progress vector information and position information of the particle species data using the created rotation matrix. After the rotation of the therapeutic device is considered, the particle species data is written and stored as the particle species data downstream of the MLC (S310).

The steps from step S304 to step S310 are performed on all the particles between control points (S311). The steps are repeated for all inter-control points (S312) to create the particle species data downstream of the MLC.

The calculated dose distribution is displayed on the display unit 122 as a dose distribution not only in a conventional manner, but also in a parallel manner or a superimposed manner allowing comparison with the dose distribution at the time of treatment planning. The display unit 122 is not limited to display only the dose distribution, and can display a y index and a DVH in addition to or instead of the dose distribution.

Display of the dose distribution is not limited to a mode displayed on the display unit 122 in the dose distribution calculation system 113, and can be a mode displayed on a display unit (not illustrated for convenience of illustration) at another location.

Here, influence of Compton scattering in the MLC can be considered to improve calculation accuracy of the MLC transport calculation.

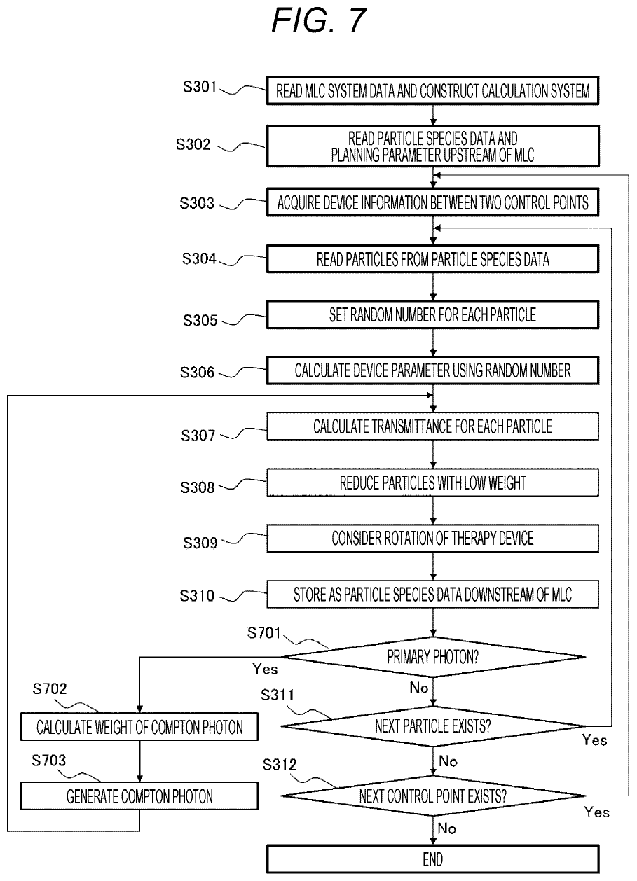

In this case, a step of generating photons (Compton photons) scattered by Compton scattering is provided after step S310 as illustrated in FIG. 7.

More specifically, the MLC transport calculation unit 120 checks particle species to determine whether it is a primary photon (S701). When it is determined as the primary photon, subsequently, a weight of the Compton photon is calculated (S702), and the Compton photon is generated (S703). After that, the processing returns to step S307 to perform the processing from step S307 also on the Compton photons. When the particle species is not determined to be the primary photon, the processing may proceed to step S311.

Although the dose distribution calculation unit 121 has been described in which a statistical weight is used in step S205 to calculate a dose distribution using transmittance, the dose distribution calculation unit 121 can calculate a dose by applying the Russian roulette method with a cut-off value of 1 without directly using a statistical weight in the dose distribution calculation to create particle species data from which particles with a low weight are deleted, the particle species data being created at a position downstream of the MLC.

Next, effects of the present embodiment will be described.

The dose distribution calculation system 113 calculates a dose distribution of radiation irradiated through the multi-leaf collimator of the present embodiment described above, the system including: the MLC transport calculation unit 120 (transport calculation procedure, transport calculation step) that calculates transmission paths through the multi-leaf collimator on two projection planes for each of particles of radiation to calculate transmittance for each of the particles; and the dose distribution calculation unit 121 (dose distribution calculation procedure, dose distribution calculation step) that calculates a dose distribution using the transmittance.

This kind of configuration enables efficient calculation of particle species data (Phase space data) downstream of the MLC, the particle species data reproducing VMAT and DWA with high accuracy, which are each an irradiation method for continuously extracting beams from multiple directions, and thus obtaining an effect of enabling a calculation result of a dose distribution with high accuracy to be efficiently obtained.

The projection plane in the leaf placement direction of the multi-leaf collimator and the projection plane in the leaf moving direction thereof serve as the two projection planes, so that the transmission path through the multi-leaf collimator can be calculated more easily, and the particle species data can be calculated more efficiently.

The MLC transport calculation unit 120 acquires a path length from a common range of transmission paths calculated on the two projection planes for each of particles of the radiation to obtain the transmittance, so that the path length can be obtained by simpler calculation, and thus a calculation load can be further reduced.

The MLC transport calculation unit 120 also performs steps of: interpolating device information at each control point using a common interpolation parameter set for each of particles of the radiation; setting a different device parameter for each of the particles of the radiation; generating particle species information in consideration of the device information that continuously changes; and acquiring a path length using the generated particle species information, so that the device information different for each of the particles can be calculated by interpolating not only discrete gantry angles but also gantry angles therebetween to consider a ring angle and a leaf position of the multi-leaf collimator at the time of irradiation at the gantry angles, and thus the dose distribution can be calculated with accuracy higher than before. Additionally, an effect is obtained in which a dose distribution can be calculated in a short time.

When the interpolation parameter is any one of a random number, a uniform random number, and a quasi-random number, calculation of a dose distribution in a short time can be achieved while accuracy is ensured.

The MLC transport calculation unit 120 also can consider influence of a dose in a region other than an irradiation field by generating Compton photons as particles and calculating transmittance of the Compton photons, and thus enables improvement in calculation accuracy of a dose distribution outside the irradiation field.

The dose distribution calculation unit 121 can provide a dose distribution in consideration of an opening shape of the MLC by directly using a statistical weight or applying the Russian roulette method when calculating the dose distribution using transmittance.

When the display unit 122 is further provided to display a dose distribution calculated by the dose distribution calculation unit 121, dose verification work of replanned treatment planning can be quickly performed based on the dose distribution obtained with high accuracy and in a short time, and thus the online adaptive treatment can be further improved in accuracy.

### <Others>

The present invention is not limited to the above embodiments, and enables various modifications and applications. The embodiments described above have been described in detail to describe the present invention in an easy-to-understand manner, and are not necessarily limited to those having all the configurations described.

For example, another aspect is provided to solve the problems of PTL 1 and NPLs 1 and 2 described above, in which device information that continuously changes is less likely to be considered in calculation as in calculation for a device that continuously moves, the calculation in consideration of a finer gantry angle, a ring angle, and an opening of an MLR, being too complicated and less likely to be performed. The other aspect is (1) a dose distribution calculation system that calculates a dose distribution of radiation irradiated through a multi-leaf collimator, the dose distribution calculation system including: a transport calculation unit that calculates transmittance for each of particles of the radiation; and a dose distribution calculation unit that calculates the dose distribution using the transmittance, the transport calculation unit being configured to perform steps of: interpolating device information at each control point using a common interpolation parameter set for each of the particles of the radiation; setting a different device parameter for each of the particles of the radiation; generating particle species information in consideration of the device information that continuously changes; and acquiring a path length of the radiation passing through the multi-leaf collimator using the generated particle species information.

The dose distribution calculation system of item (1) enables the device information different for each of the particles to be calculated by interpolating not only discrete gantry angles but also gantry angles therebetween to consider a ring angle and a leaf position of the multi-leaf collimator at the time of irradiation at the gantry angles, and thus the dose distribution can be calculated with accuracy higher than before. Additionally, the device information at the control point is interpolated with the same interpolation parameter based on the interpolation parameter set for each of the particles, so that an effect is obtained in which a dose distribution can be calculated in a short time.

(2) The dose distribution calculation system described in item (1) is configured such that the interpolation parameter is any one of a random number, a uniform random number, and a quasi-random number.

(3) The dose distribution calculation system described in item (1) or (2) is configured such that the transport calculation unit calculates transmission paths through the multi-leaf collimator on two projection planes for each of the particles of the radiation to calculate transmittance for each of the particles.

(4) The dose distribution calculation system described in item (3) is configured such that the two projection planes serve as a projection plane in a leaf placement direction of the multi-leaf collimator and a projection plane in a leaf moving direction.

(5) The dose distribution calculation system described in item (3) or (4) is configured such that the transport calculation unit acquires a path length from a common range of the transmission paths calculated on the two projection planes for each of the particles of the radiation to obtain the transmittance.

(6) The dose distribution calculation system described in any one of items (1) to (5) is configured such that the transport calculation unit generates Compton photons as the particles and calculates transmittance of the Compton photons.

(7) The dose distribution calculation system described in any one of items (1) to (6) is configured such that the dose distribution calculation unit uses a statistical weight directly or by applying a Russian roulette method when calculating the dose distribution using the transmittance.

(8) The dose distribution calculation system described in any one of items (1) to (7) is configured to further include a display unit that displays the dose distribution calculated by the dose distribution calculation unit.

### REFERENCE SIGNS LIST

A irradiation target
100 X-ray irradiation device
101 ring type gantry
102 irradiation nozzle
103 couch
104 radiation irradiation control device
105 communication device
106 storage device
107 input device
110 X-ray irradiation system
111 data server
111A treatment planning information
112 treatment planning device
113 dose distribution calculation system
120 MLC transport calculation unit
121 dose distribution calculation unit
122 display unit
123 input unit
124 storage unit
125 particle species information data base

1. A dose distribution calculation system for calculating a dose distribution of radiation irradiated through a multi-leaf collimator, the system comprising:
a transport calculation unit for calculating transmission paths through the multi-leaf collimator on two projection planes for each of particles of the radiation to calculate transmittance for each of the particles; and
a dose distribution calculation unit for calculating the dose distribution using the transmittance.

2. The dose distribution calculation system according to claim 1, wherein two projection planes serve as a projection plane in a leaf placement direction of the multi-leaf collimator and a projection plane in a leaf moving direction.

3. The dose distribution calculation system according to claim 1, wherein the transport calculation unit acquires a path length from a common range of the transmission paths calculated on two projection planes for each of the particles of the radiation to obtain the transmittance.

4. The dose distribution calculation system according to claim 3, wherein
the transport calculation unit performs steps of:
interpolating device information at each control point using a common interpolation parameter set for each of the particles of the radiation;
setting a different device parameter for each of the particles of the radiation;
generating particle species information in consideration of the device information that continuously changes; and
acquiring the path length using the generated particle species information.

5. The dose distribution calculation system according to claim 4, wherein the interpolation parameter is any one of a random number, a uniform random number, and a quasi-random number.

6. The dose distribution calculation system according to claim 1, wherein the transport calculation unit generates Compton photons as the particles and calculates transmittance of the Compton photons.

7. The dose distribution calculation system according to claim 1, wherein the dose distribution calculation unit uses a statistical weight directly or by applying a Russian roulette method when calculating the dose distribution using the transmittance.

8. The dose distribution calculation system according to claim 1, further comprising a display unit that displays the dose distribution calculated by the dose distribution calculation unit.

9. A program that causes a calculation system to perform procedures, the procedures comprising:
a transport calculation procedure of calculating transmission paths through a multi-leaf collimator on two projection planes for each of particles of radiation to calculate transmittance for each of the particles; and
a dose distribution calculation procedure of calculating a dose distribution of the radiation irradiated through the multi-leaf collimator using the transmittance.

10. A method for calculating a dose distribution of radiation irradiated through a multi-leaf collimator, the method comprising:
a transport calculation step of calculating transmission paths through the multi-leaf collimator on two projection planes for each of particles of radiation and calculating transmittance for each of the particles; and
a dose distribution calculation step of calculating the dose distribution using the transmittance.
